(19) United States
(12) Patent Application Publication
(10) Pub. No.: US 2024/0194348 A1
(43) Pub. Date: Jun. 13, 2024

(54) Medical Support System and Medical Support Method for Patient Treatment

(71) Applicant: BIOTRONIK SE & Co. KG, Berlin (DE)

(72) Inventors: Thomas DOERR, Berlin (DE); Jens MUELLER, Berlin (DE); Matthias GRATZ, Erlangen (DE); R. Hollis WHITTINGTON, Portland, OR (US)

(73) Assignee: BIOTRONIK SE & Co. KG, Berlin (DE)

(21) Appl. No.: 18/553,643

(22) PCT Filed: Mar. 17, 2022
(86) PCT No.: PCT/EP2022/056962

§ 371 Date: Oct. 2, 2023

Related U.S. Application Data
(60) Provisional application No. 63/172,114, filed on Apr. 8, 2021.

(30) Foreign Application Priority Data
May 3, 2021 (EP) 21171773.1

(51) Int. Cl. G16H 50/20; G16H 10/60; G16H 20/40; G16H 50/70

(52) U.S. Cl. CPC G16H 50/20; G16H 10/60; G16H 20/40; G16H 50/70

(57) ABSTRACT

A medical support system for patient treatment. The medical support system includes a central computing entity; and at least one local medical entity configured to obtain physiological data of a patient, preprocess the physiological data to generate anonymous preprocessed data, and send the anonymous preprocessed data to the central computing entity via at least one communications network, wherein the central computing entity is configured to analyze the anonymous preprocessed data, determine one or more parameters in relation to a medical support function based on the analysis of the anonymous preprocessed data, and send the determined one or more parameters to the at least one local medical entity via the at least one communications network, and wherein the at least one local medical entity is configured to implement the one or more parameters received from the central computing entity to provide the medical support function for the patient.

### CROSS-REFERENCE TO RELATED APPLICATIONS

This application is the United States National Phase under 35 U.S.C. § 371 of PCT International Patent Application No. PCT/EP2022/056962, filed on Mar. 17, 2022, which claims the benefit of European Patent Application No. 21171773.1, filed on May 3, 2021, and U.S. Provisional Patent Application No. 63/172,114, filed on Apr. 8, 2021, the disclosures of which are hereby incorporated by reference herein in their entireties.

### TECHNICAL FIELD

Embodiments of the present disclosure relate to a medical support system for patient treatment, a medical support method for patient treatment, and a machine readable medium to execute the medical support method. Embodiments of the present disclosure relate more particularly to artificial intelligence (AI)-assisted real-time medical diagnostics and/or artificial intelligence (AI)-assisted real-time medical procedures that provide data protection.

### BACKGROUND

In today's healthcare delivery, for example, in hospitals and through ambulance services, it is increasingly difficult to find the optimal treatment for a patient. In particular, there is an increasing challenge to identify the most effective treatment for a patient as well as to reduce costs and achieve greater efficiency. These difficulties are due, at least in part, to rising cost pressures, an increasing number of treatment options, and increased information availability.

To address at least the above challenges, electronic medical support systems have recently been developed. However, patient data is generally subject to privacy protection, which limits the potential applications of electronic medical support systems.

In light of the above, a medical support system for patient treatment, a medical support method for patient treatment, and a machine readable medium to execute the medical support method are provided.

The present disclosure is directed toward overcoming one or more of the above-mentioned problems, though not necessarily limited to embodiments that do.

### SUMMARY

It is an object of the present disclosure to provide improved medical diagnostics and/or medical procedures while ensuring privacy. It is another object of the present disclosure to provide real-time support for medical diagnostics and/or medical procedures.

At least the objects are solved by the features of the independent claims. Preferred embodiments are defined in the dependent claims.

According to an independent aspect of the present disclosure, a medical support system for patient treatment is provided. The medical support system includes a central computing entity: and at least one local medical entity. The at least one local medical entity is configured to obtain (e.g., real-time) physiological data of a patient, preprocess the physiological data to generate anonymous preprocessed data, and send the anonymous preprocessed data to the central computing entity via at least one communications network. The central computing entity is configured to analyze the anonymous preprocessed data, determine one or more parameters in relation to a medical support function based on the analysis of the anonymous preprocessed data, and send the determined one or more parameters to the at least one local medical entity via the at least one communications network. The at least one local medical entity is configured to implement the one or more parameters received from the central computing entity to provide the medical support function for the patient.

For example, the at least one local medical entity may perform the local preprocessing of the physiological data to determine technical parameters relating to an execution of the medical support function, such as an automated cardiac ablation procedure. The central computing entity may then determine procedure control parameters for controlling the automated cardiac ablation procedure based on the technical parameters received from the at least one local medical entity. This allows a real-time medical support function to be delivered using a distributed computing architecture, while still complying with privacy regulations.

According to some embodiments, which can be combined with other embodiments described herein, the central computing entity is a cloud computing entity (cloud or cloud system). Additionally, or alternatively, the at least one local medical entity is an edge computing entity (edge node or edge device).

According to some embodiments, which can be combined with other embodiments described herein, the at least one local medical entity and the central computing entity may be configured to communicate in real-time to provide the medical support function.

According to some embodiments, which can be combined with other embodiments described herein, the central computing entity is configured to analyze the anonymous preprocessed data using an artificial intelligence algorithm. In particular, the central computing entity may include at least one artificial intelligence module or AI-based data processing unit configured to analyze the anonymous preprocessed data using an artificial intelligence algorithm.

According to some embodiments, which can be combined with other embodiments described herein, the artificial intelligence algorithm is a machine learning algorithm. In some implementations, the machine learning algorithm may be a neural network, such as a feed-forward network, a recurrent neural network, or a convolutional neural network.

According to some embodiments, which can be combined with other embodiments described herein, the artificial intelligence algorithm is trained based on anonymous physiological patient data.

According to some embodiments, which can be combined with other embodiments described herein, the central computing entity is configured to provide a software component (e.g., a particular component of the artificial intelligence algorithm) to the at least one local medical entity. The at least one local medical entity may be configured to generate the anonymous preprocessed data using the software component received from the central computing entity.

According to some embodiments, which can be combined with other embodiments described herein, the at least one local medical entity is configured to request the software component, such as a particular component of the artificial intelligence algorithm, from the central computing entity, according to a medical support function to be provided to the patient.

According to some embodiments, which can be combined with other embodiments herein, the central computing entity is configured to provide a software component to the at least one local medical entity, and wherein the at least one local medical entity is configured to generate the anonymous preprocessed data using the software component, wherein the at least one local medical entity is configured to request the software component from the central computing entity according to a medical support function to be provided to the patient.

This procedure makes it possible for (confidential) patient data to be pre-processed locally and then further processed in the cloud, but freed from personal data (for privacy reasons).

According to some embodiments, which can be combined with other embodiments described herein, the at least one local medical entity is configured to encrypt the anonymous preprocessed data and send the encrypted anonymous preprocessed data to the central computing entity.

According to some embodiments, which can be combined with other embodiments described herein, the central computing entity is configured to encrypt the one or more parameters and send the encrypted one or more parameters to the at least one local medical entity.

According to some embodiments, which can be combined with other embodiments described herein, the at least one communications network includes a local network and/or a mobile network.

Preferably, the mobile network is a 5G mobile network.

According to some embodiments, which can be combined with other embodiments described herein, a latency of the at least one communications network is 10 ms or less.

According to some embodiments, which can be combined with other embodiments described herein, a bandwidth provided by the at least one communications network is 100 Mbit/s or higher.

According to some embodiments, which can be combined with other embodiments described herein, the medical support function may be selected from the group including (or consisting of) medical diagnostics, medical decision-making, medical treatment of the patient, and combinations thereof.

According to some embodiments, which can be combined with other embodiments described herein, the medical support system may be configured for cardiac disease diagnostics and/or cardiac disease treatment and/or electrophysiology and/or medical virtual reality procedures and/or medical enhanced reality procedures and/or robot-assisted surgery and/or neurological treatment/stimulation and/or combinations thereof.

According to some embodiments, which can be combined with other embodiments described herein, the at least one local medical entity and the central computing entity are configured to communicate in real-time to provide the medical support function.

According to some embodiments, which can be combined with other embodiments described herein, the anonymous preprocessed data include one or more technical parameters derived from the physiological data of the patient and relating to an execution of the medical support function. The technical parameters may be non-protected technical parameters, i.e., information that comply with data privacy regulations. For example, in the case of an automated cardiac ablation procedure, the technical parameters may include a lesion position and/or a lesion depth derived from, for example, an electroanatomic map of the patient.

According to some embodiments, which can be combined with other embodiments described herein, the one or more parameters determined by the central computing entity are procedure control parameters for controlling the medical support function. In particular, the procedure control parameters may be determined based on the one or more technical parameters provided by the at least one local medical entity. For example, in the case of an automated cardiac ablation procedure, the procedure control parameters may be an energy and/or a duration of the ablation process.

According to another independent aspect of the present disclosure, a medical support method for patient treatment is provided. The medical support method includes acquiring, by at least one local medical entity, (e.g., real-time) physiological data of a patient: preprocessing, by the at least one local medical entity, the physiological data to generate anonymous preprocessed data: sending, by the at least one local medical entity, the anonymous preprocessed data to a central computing entity via at least one communications network: analyzing, by the central computing entity, the anonymous preprocessed data: determining, by the central computing entity, one or more parameters in relation to a medical support function based on the analysis of the anonymous preprocessed data: sending, by the central computing entity, the determined one or more parameters to the at least one local medical entity via the at least one communications network: and implementing, by the at least one local medical entity, the one or more parameters received from the central computing entity to provide the medical support function for the patient.

Embodiments are also directed at systems for carrying out the disclosed methods and include system aspects for performing each described method aspect. These method aspects may be performed by way of hardware components, a computer programmed by appropriate software, by any combination of the two or in any other manner. Furthermore, embodiments according to the present invention are also directed at methods for operating the described system. It includes method aspects for carrying out every function of the medical support system.

According to another independent aspect of the present disclosure, a machine-readable medium is provided. The machine-readable medium includes instructions executable by one or more processors to implement the medical support method for patient treatment of the embodiments of the present disclosure.

The (e.g., non-transitory) machine readable medium may include, for example, optical media such as CD-ROMs and digital video disks (DVDs), and semiconductor memory devices such as Electrically Programmable Read-Only Memory (EPROM), and Electrically Erasable Programmable Read-Only Memory (EEPROM). The machine-readable medium may be used to tangibly retain computer program instructions or code organized into one or more modules and written in any desired computer programming language. When executed by, for example, one or more processors such computer program code may implement one or more of the methods described herein.

According to another independent aspect of the present disclosure, a medical support system for patient treatment is provided. The medical support system includes one or more processors: and a memory (e.g., the above machine-readable medium) coupled to the one or more processors and comprising instructions executable by the one or more processors to implement the medical support method for patient treatment of the embodiments of the present disclosure.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

So that the manner in which the above recited features of the present disclosure can be understood in detail, a more particular description of the disclosure, briefly summarized above, may be had by reference to embodiments. The accompanying drawings relate to embodiments of the disclosure and are described in the following:

FIG. 1 shows a schematic view of a medical support system for patient treatment according to embodiments described herein:

FIG. 2 shows a schematic view of a medical support system for patient treatment according to further embodiments described herein; and

FIG. 3 shows a flow chart of a medical support method for patient treatment according to embodiments described herein.

### DETAILED DESCRIPTION

Reference will now be made in detail to the various embodiments of the disclosure, one or more examples of which are illustrated in the figures. Within the following description of the drawings, the same reference numbers refer to same components. Generally, only the differences with respect to individual embodiments are described. Each example is provided by way of explanation of the disclosure and is not meant as a limitation of the disclosure. Further, features illustrated or described as part of one embodiment can be used on or in conjunction with other embodiments to yield yet a further embodiment. It is intended that the description includes such modifications and variations.

Nowadays it is increasingly difficult to find the optimal treatment for a patient. These difficulties are due, at least in part, to rising cost pressures, an increasing number of treatment options, and increased information availability. Further, patient data is generally subject to privacy protection, which limits the potential applications of electronic medical support systems.

The embodiments of the present disclosure address the above challenges by implementing a distributed computing architecture in which a local computing entity and a central computing entity work together to provide a medical support function in real-time. In particular, the local computing entity collects and preprocesses patient data, and sends the preprocessed and now anonymous patient data to the central computing entity for further processing. Thereby, the medical support function can be provided in real-time while ensuring privacy. Specifically, protected patient data, such as the patient's identity, do not leave a protected environment, while the considerable computing resources of the central computing entity can still be used to provide the medical support function in real-time.

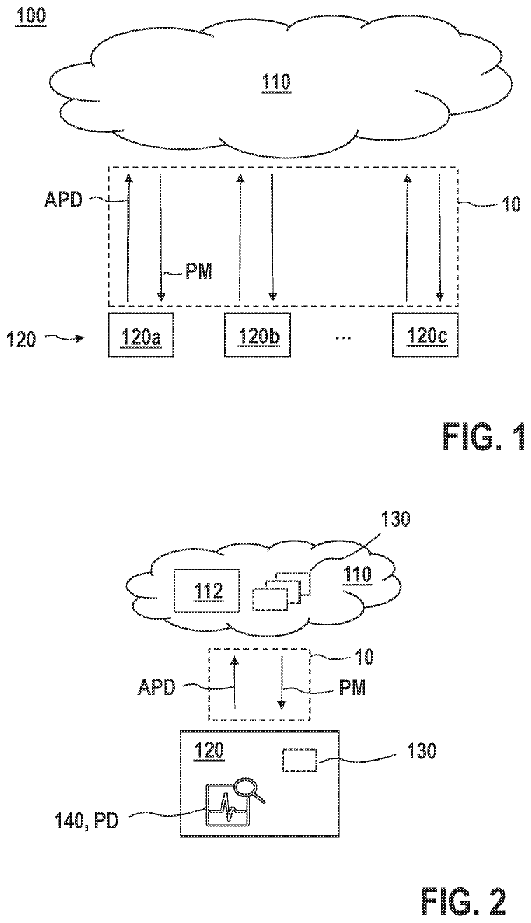

FIG. 1 shows a schematic view of a medical support system 100 for patient treatment according to embodiments described herein.

The medical support system 100 includes a central computing entity 110: and at least one local medical entity 120 configured to obtain (e.g., real-time) physiological data PD of a patient, preprocess the physiological data PD to generate anonymous preprocessed data APD, and send the anonymous preprocessed data APD to the central computing entity 110 via at least one communications network 10. The central computing entity 110 is configured to analyze the anonymous preprocessed data APD, determine one or more parameters PM in relation to a medical support function based on the analysis of the anonymous preprocessed data APD, and send the determined one or more parameters PM to the at least one local medical entity 120 via the at least one communications network 10. The at least one local medical entity 120 is configured to implement the one or more parameters PM received from the central computing entity 110 to provide the medical support function for the patient.

According to some embodiments, which can be combined with other embodiments described herein, the central computing entity 110 is a cloud computing entity (cloud or cloud system), and the at least one local medical entity 120 is an edge computing entity (edge node or edge device). Such edge computing is distributed computing that can improve response times and save bandwidth, enabling a real-time medical support function.

In the example of FIG. 1, the at least one local medical entity 120 includes a plurality of local medical entities 120a, 120b and 120c. Each local medical entity is a unit or system provided at a patient's and/or doctor's site, such as inside a hospital setting. The local medical entity may preprocess real-time patient data locally (patient site: edge), and only the preprocessed anonymous data are sent to, and used in, the central computing entity 110 (cloud). This distributed computing architecture ensures data privacy by separation of patient data (in local IT at acquisition site, e.g., a hospital) and centralized data processing.

The term “anonymous” means that the preprocessing is performed such that the identity of the patient cannot be derived anymore from the data sent to the central computing entity 110.

The physiological data of the patient collected by the at least one local medical entity 120 may be any data regarding an actual and/or general physiological state of the patient. For example, the physiological data may include real-time measurement data (e.g., a heart rate, blood pressure, ECG, etc.), an age of the patient, relevant vital data, examination results, etc.

In some embodiments, the physiological data may include one or more physiological parameters of the patient. The one or more physiological parameters can be selected from the group including (or consisting of) heart rate, blood pressure, body temperature, and serum levels (e.g., of various stress hormones). However, the present disclosure is not limited thereto, and other physiological parameters that are useful to provide the medical support function may be obtained or measured by the at least one local medical entity 120.

The at least one local medical entity 120 and the central computing entity 110 communicate with each other via the at least one communications network 10 to exchange data. The data exchange includes at least the anonymous preprocessed data APD as well as the one or more parameters PM in relation to the medical support function.

The at least one communications network 10 may include a mobile network and/or a local network. Preferably, the mobile network is a 5G mobile network but is not limited thereto.

The mobile network 10 may use any of various wireless communication technologies, or telecommunication standards, such as GSM, UMTS, LTE, LTE-Advanced (LTE-A), 5G, HSPA, and the like. For example, the at least one local medical entity 120 may include a communication module configured to implement a communication profile such as an embedded subscriber identification module, eSIM, profile. However, the present disclosure is not limited thereto, and a conventional SIM may be used or another non-SIM communication profile.

The local network may use any of various wired and/or wireless communication technologies, such as Local Area Networks (LANs), Wireless LAN (WiFi), Bluetooth, and the like.

It is to be understood that two or more different types of communications networks can be used in combination to enable the combination between the central computing entity 110 and the at least one local medical entity 120. For example, the at least one local medical entity 120 may be connected to a local network to communication within a local environment, such as a hospital setting. A suitable interface may be provided to allow data transfer between the local network and a mobile network, such as a 5G network, to provide the anonymous preprocessed data APD to the central computing entity 110.

In some implementations, a latency of the at least one communications network 10 is 10 ms or less. In other words, the communications network 10 may be a low-latency communications network. The term “real-time”, as it is used throughout the present application, means that data are collected, processed, and communicated over such low-latency communications networks. In particular, the term “real-time” means that (medical) workflows, e.g., during a surgical procedure can be carried out continuously without interruptions or delays.

According to some embodiments, which can be combined with other embodiments described herein, the at least one local medical entity 120 and the central computing entity 110 can directly communicate with each other via the at least one communications network 10. In other embodiments, the at least one local medical entity 120 and the central computing entity 110 can indirectly communicate with each other via one or more further entities, such as further intermediate local and/or central computing entities.

In some embodiments, the medical support function is a medical diagnostics function, a medical decision-making function, or a medical treatment function. For example, the medical support system 100 may be configured for cardiac disease diagnostics, cardiac disease treatment, electrophysiology, medical virtual reality (VR) procedures, medical enhanced reality (ER) procedures, and/or robot-assisted surgery.

In one example, the medical support system 100 can be configured for electrophysiology. Electrophysiology pertains to electrical recording techniques that enable the measurement of electrical properties of biological tissue using, for example, electrodes. The at least one local medical entity 120 and the central computing entity 110 may communicate in real-time to control and perform the measurement of the electrical properties. For example, the one or more parameters PM determined by the central computing entity 110 may be control parameters to control the measurement of the electrical properties of the biological tissue.

In another example, the medical support system 100 can be configured for medical virtual reality procedures and/or medical enhanced reality procedures. Medical virtual reality systems can simulate a surgical procedure, e.g., in order to improve training of medical staff. Enhanced reality systems can augment or enhance reality, e.g., by providing improved imaging.

In yet another example, the medical support system 100 can be configured for conducting and/or assisting surgical procedures. For example, the medical support system 100 can be configured for robotic surgery or robot-assisted surgery. Robotic surgery or robot-assisted surgery is a type of surgical procedure that is done using robotic systems. In this case, the one or more parameters PM determined by the central computing entity 110 may be control parameters to control various automated devices, such as robotic surgical devices, configured for robotic surgery or robot-assisted surgery.

For example, the at least one local medical entity 120 may perform the local preprocessing of the physiological data PD to determine technical parameters relating to an execution of the robotic surgery or robot-assisted surgery, such as an automated cardiac ablation procedure. The central computing entity 110 may then determine procedure control parameters for controlling the automated cardiac ablation procedure based on the technical parameters received from the at least one local medical entity 120. For example, the procedure control parameters may be an energy and/or a duration of the ablation process. This allows a real-time medical support function to be delivered using a distributed computing architecture, while still complying with privacy regulations.

According to further or alternative embodiments, the at least one local computing entity 120 may include at least one output device. The medical support system 100 may be configured to control the at least one output device based on the one or more parameters PM received from the central computing entity 110 to output information indicative of the analysis of the anonymous preprocessed data APD. For example, the at least one output device may include at least one display device and/or at least acoustical device (e.g., at least one loudspeaker).

In some embodiments, the output information provided by the at least one output device may include, or be, a medical diagnosis and/or medical treatment instructions. The output information may be, for example, instructions for the administration of a drug by medical personnel.

According to some embodiments, which can be combined with other embodiments described herein, the at least one local computing entity 120 further includes a control module (not shown) configured to control one or more medical treatment devices based on the one or more parameters PM received from the central computing entity 110. An example of a medical treatment device is a device for drug administration or automatic drug administration. The one or more parameters PM received from the central computing entity 110 may indicate a type of the drug, a dosage of the drug, a timing of the automatic drug administration, and the like.

In some implementations, the control module of the medical support system 100 is configured to initiate and/or change a treatment of the patient by the control of the one or more medical treatment devices. For example, the control module may change a drug dose administered to the patient based on the one or more parameters PM received from the central computing entity 110.

FIG. 2 shows a schematic view of a medical support system for patient treatment according to further embodiments described herein. The medical support system is similar to the medical support system of FIG. 1 and therefore, a description of identical or similar features is not repeated.

The at least one local medical entity 120 can be configured to obtain real-time physiological data PD of the patient, such as one or more physiological parameters. The physiological data PD is preprocessed at the patient's site to anonymize the physiological data PD. The anonymized physiological data APD is sent to the central computing entity 110 for further processing to provide the medical support function. In particular, the central computing entity 110 is configured to analyze the anonymous preprocessed data APD for determining one or more parameters PM in relation to a medical support function.

According to some embodiments, which can be combined with other embodiments described herein, the at least one local medical entity 120 includes, or is, e.g., wired or wirelessly connected to one or more sensors 140 associated with the patient. The one or more sensors 140 may be configured to detect the one or more physiological parameters.

The one or more sensors 140 can be configured for non-invasive and/or invasive monitoring. For example, the one or more sensors 140 can be connected or attached to the patient's body to determine or monitor the physiological parameters in an invasive or a non-invasive manner.

According to some embodiments, which can be combined with other embodiments described herein, the at least one local medical entity 120 is configured to encrypt the anonymous preprocessed data APD and send the encrypted anonymous preprocessed data to the central computing entity 110. The encryption may use Blockchains or another suitable encryption scheme.

Additionally, or alternatively, the central computing entity 110 may be configured to encrypt the one or more parameters PM and send the encrypted one or more parameters to the at least one local medical entity 120 for implementing, e.g., robot-assisted treatment of the patient.

The central computing entity 110 may include at least one artificial intelligence module 112 or AI-based data processing unit configured to analyze the anonymous preprocessed data APD using an artificial intelligence algorithm.

The term “artificial intelligence” as used throughout the present application may be understood in the sense of software components or software instances which are designed to correctly interpret data (i.e., the anonymous preprocessed data), to learn from such data, and to use those learnings to provide a medical support function through flexible adaptation.

In some embodiments, the artificial intelligence algorithm may be trained based on anonymous physiological patient data.

According to some embodiments, the artificial intelligence algorithm is a machine learning algorithm, in particular a neural network. The neural network may be a feed-forward network with multiple hidden layers, a recurrent neural network, or a convolutional neural network with three dimensions.

A neural network is based on a collection of connected nodes. A node that receives a signal processes it and can signal nodes connected to it. Typically, nodes are aggregated into layers. Different layers may perform different transformations on their inputs. Signals travel from an input layer to an output layer. Such a neural network may be trained by processing examples, each of which contains a known input and result, forming probability-weighted associations between the two, which are stored within the data structure of the neural network. Thus, the neural network learns to perform tasks by considering examples.

According to some embodiments, which can be combined with other embodiments described herein, the central computing entity 110 is configured to instruct the at least one local medical entity 120 how to perform the preprocessing of the patient data. For example, the central computing entity 110 may be configured to provide a software component 130 to the at least one local medical entity 120. The at least one local medical entity 120 may be configured to generate the anonymous preprocessed data APD using the software component 130.

In some implementations, the at least one local medical entity 120 may be configured to request the software component 130 from the central computing entity 110. For example, the at least one local medical entity 120 may request the provision of a particular medical support function, and the central computing entity 110 may send a corresponding software component 130 to the at least one local medical entity 120. The software component 130 may indicate or define a type and/or a content and/or a format of data required by the central computing entity 110 to carry out a particular medical support function.

All communication between the at least one local medical entity 120 (edge system) and the central computing entity 110 (cloud system) is carried out via respective communication units that can establish and maintain a secure connection within or with a scalable real-time capable IoT infrastructure. An example of this is 5G technology. Communication between the at least one local medical entity 120 and the central computing entity 110 may be direct or indirect using, for example, intermediary cloud infrastructures.

The central computing entity 110 can be operated in a decentralized manner using scalable real-time IoT infrastructures (cloud services, Saas, etc.) due to regional regulatory requirements. Therefore, it is possible for edge systems in one country, for example, to communicate with locally operated cloud systems, while edge systems in other countries access cloud systems outside said one country. The communication units forward the information to a processing unit (not shown) of the edge system 120 and the AI-based data processing unit 112 in the cloud system 110, respectively. The transmitted information units, in addition to security mechanisms such as those provided by 5G network technology, may be protected against falsification and misuse at the business layer level by means of blockchain or comparable methods.

As mentioned above, the cloud system 110 may include an AI-based data processing unit 112 trained for medical decisions, diagnostics and/or therapy management by machine. The AI-based data processing unit 112 may be configured to implement a machine learning algorithm in the form of a neural network. Deep learning may be used to train the neural network. Preferably, the neural network is a feed-forward network with multiple hidden layers or a recurrent neural network with multiple hidden layers. If a diagnostic therapeutic procedure requires, for instance, processing of image data, video data, or audio data, it is preferable to use a neural network with more than 2 dimensions (e.g., a convolutional neural network with 3 dimensions).

The AI based data processing unit 112 may optionally include input signal conditioning (filtering, scaling, normalization, transformation, etc.) and/or result post-processing (clustering, weighting, filtering, plausibility checking, etc.).

Optionally, the neural network may have a model control layer to make the medical application traceable (verification and monitoring of the model). The type of data used to train the neural network may depend largely on the desired medical support function. In any case, preprocessed data could be used for training the neural network (anonymous training).

The edge system 120 is applied to the patient by the physician and acquires, among other things, physiological data in real-time. This edge system 120 is also configured to support—in combination with the cloud system 110—medical decisions, diagnostics and therapy control by machine.

Since the edge system 120 collects confidential patient data that must not be transmitted to the cloud system 110 for privacy reasons, the latter may request specific algorithm parts (e.g., the software component 130) from the cloud system 110 in real-time, which specific algorithm parts 130 can be executed to preprocess the patient data in the edge system 120. The results of this preprocessing, which are free of protected patient data, are then transmitted to the cloud system 110 for further processing in real-time and processed, e.g., in an AI-based core process. The results are transmitted to the edge system 120 in real-time and used there for diagnostics and/or therapy management. The whole process can be repeated until the procedure is finished by one of the two participants.

In some embodiments, the specific algorithm parts 130 may be obtained only initially and/or once by the edge system 120 from the cloud system 110. The entire data flow (e.g., transfer of the specific algorithm parts, transmission of technical procedure parameters, results of diagnostics and therapy control, etc.) may be transmitted via a secured channel over the at least one communications network 10.

Below are two application examples given in more detail.

### Automated Cardiac Ablation Procedure

The physician-side data acquisition and processing system (edge system 120), in this case a hospital ablation system, may request one or more specific algorithms 130 from the cloud system 110 (centralized medical decision system) for the cardiac ablation procedure to calculate technical output parameters from the physiological input parameters PD.

For example, the edge system 120 can process at least one of the following physiological input parameters PD using the specific algorithms 130: electroanatomical map(s), ECG, arrhythmia documentation (EPU examination), general patient data (e.g., age, indication for ablation, laboratory values, other procedure-relevant vital data, etc.), and catheter measurements (e.g., impedance, temperature, force, etc.).

The edge system 120 can use the specific algorithm 130 to locally preprocess the physiological input parameters PD to calculate, for example, technical output parameters that are not protected, such as lesion positions and lesion depth(s).

The technical output parameters are sent to the cloud system 110 and forwarded to the AI-based data processing unit 112 within the cloud system 110. The AI-based data processing unit 112 extracts the technical output parameters, processes them, and creates technical procedure parameters. In this case, the procedure parameters can be ablation parameters.

For example, the following parameters may serve as input data for the AI-based data processing unit 112 (e.g., the neural network is also trained with these parameter types): normalized electroanatomical map(s), catheter position index (“GPS data” on the electroanatomical map), technical measurement values of the catheter, technical ablation system used (technical data of the ablator such as hardware and software version and the catheter type, batch number, etc.). These values may be initially transferred from the edge system 120 to the cloud system 110.

The AI-based data processing unit 112 may calculate the following output parameters (procedure parameters, in this case ablation parameters): energy, ablation duration, impedance ranges, and force window.

The ablation parameters (procedure parameters) are transmitted by the cloud system 110 to the ablation system in the hospital (edge system 120). The entire process can be repeated until the procedure is terminated by either participant.

### Automated Transcatheter Aortic Valve Implantation (Release of a TAVI Valve/Percutaneous Aortic Valve Replacement)

The physician-side data acquisition and processing system (edge system 120), in this case a system for a hospital aortic valve replacement, requests one or more specific algorithms 130 from the centralized medical decision system (cloud system 110) for the minimally invasive procedure, in order to be able to calculate technical output parameters from the physiological input parameters PD.

By means of the specific algorithms 130, the edge system 120 can process, for example, one or more of the following protected physiological input parameters PD: CT or MRI images/movies of the heart and aorta, including the outlet of the coronary arteries, flow data/pressure gradients upstream and downstream of the valve, catheter position, and general patient data (e.g., age, indication, laboratory values, other vital data relevant to the procedure).

The edge system 120 can use the specific algorithm 130 to locally preprocess the physiological input parameters PD to calculate all the necessary non-protected technical output parameters that the cloud system 110 needs to calculate the procedure parameters to control the surgical procedure (transcatheter aortic valve implantation, or “TAVI” for short).

The technical output parameters are sent to the cloud system 110 and forwarded to the AI-based data processing unit 112 within the cloud system 110. The AI-based data processing unit 112 extracts the technical output parameters, processes them, and creates technical procedure parameters. In this case, the procedure parameters may be valve release control parameters.

The following parameters, among others, may serve as input data for the AI-based data processing unit 112 (the neural network is also trained with these parameter types): normalized CT or MRI images/movies of the heart and aorta including the outflow of the coronary arteries, normalized flow data, catheter position (“GPS data” on the electroanatomical map), and technical system used to release the TAVI valve (technical data of hardware and software, e.g., catheter type, TAVI valve, batch number, etc.). These values are initially transmitted from the edge system 120 to the cloud system 110.

The AI-based data processing unit 112 calculates the following output parameters (procedure parameters, in this case parameters for transcatheter aortic valve implantation (TAVI)): position index, expansion pressure/volume, expansion duration, and parameters for radiofrequency stimulation during expansion. The parameters for TAVI are transmitted by the cloud system 110 to the system for an aortic valve replacement in the clinic (edge system 120). The entire procedure can be repeated until the procedure is terminated by either participant.

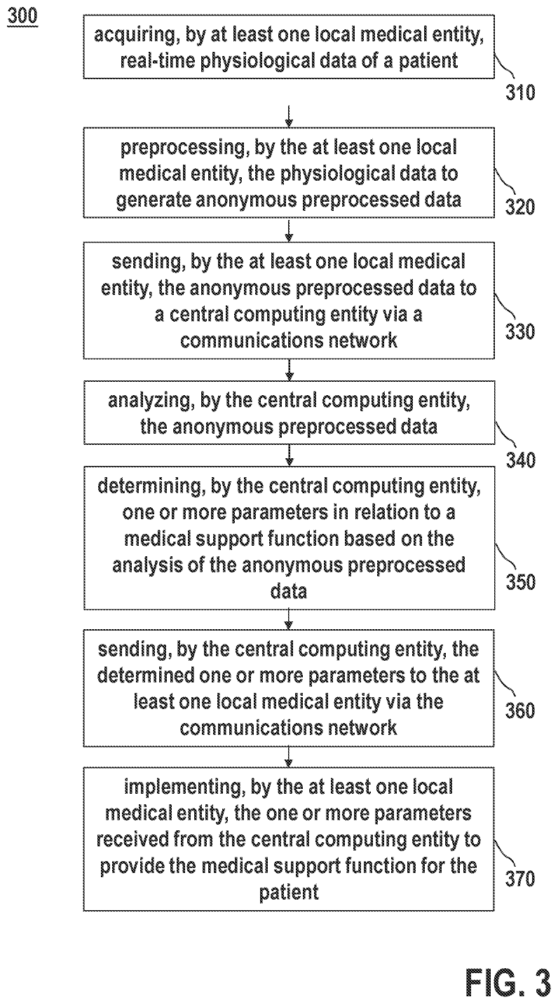

FIG. 3 shows a flow chart of a medical support method 300 for patient treatment according to embodiments described herein.

The physician-side data acquisition and processing system (edge system 120) may request one or more specific algorithms 130 from the cloud system 110 (centralized medical decision system) to calculate technical output parameters from the physiological input parameters PD.

The medical support method 300 includes in block 310 acquiring, by at least one local medical entity, real-time physiological data of a patient: in block 320 preprocessing, by the at least one local medical entity, the physiological data to generate anonymous preprocessed data: in block 330 sending, by the at least one local medical entity, the anonymous preprocessed data to a central computing entity via at least one communications network: in block 340 analyzing, by the central computing entity, the anonymous preprocessed data: in block 350 determining, by the central computing entity, one or more parameters in relation to a medical support function based on the analysis of the anonymous preprocessed data: in block 360 sending, by the central computing entity, the determined one or more parameters to the at least one local medical entity via the at least one communications network; and in block 370 implementing, by the at least one local medical entity, the one or more parameters received from the central computing entity to provide the medical support function for the patient.

According to embodiments described herein, the medical support method for patient treatment can be conducted by means of computer programs, software, computer software products and the interrelated controllers, which can have a CPU, a memory, a user interface, and input and output means being in communication with the corresponding components of the medical support system for patient treatment.

Embodiments of the present disclosure implement a distributed computing architecture in which a local computing entity and a central computing entity work together to provide a medical support function in real-time. In particular, the local computing entity collects and preprocesses patient data, and sends the preprocessed and now anonymous patient data to the central computing entity for further processing. Thereby, the medical support function can be provided in real-time while ensuring privacy. Specifically, protected patient data, such as the patient's identity, do not leave a protected environment, while the considerable computing resources of the central computing entity can still be used to provide the medical support function in real-time.

While the foregoing is directed to embodiments of the disclosure, other and further embodiments of the disclosure may be devised without departing from the basic scope thereof, and the scope thereof is determined by the claims that follow.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A medical support system for patient treatment, comprising:
a central computing entity; and
at least one local medical entity; configured to obtain physiological data of a patient, preprocess the physiological data to generate anonymous preprocessed data, and send the anonymous preprocessed data to the central computing entity via at least one communications network,
wherein the central computing entity is configured to analyze the anonymous preprocessed data, determine one or more parameters in relation to a medical support function based on the analysis of the anonymous preprocessed data, and send the determined one or more parameters to the at least one local medical entity via the at least one communications network, and wherein the at least one local medical entity is configured to implement the one or more parameters received from the central computing entity to provide the medical support function for the patient.

2. The medical support system of claim 1, wherein the central computing entity a cloud computing entity, and/or wherein the at least one local medical entity is an edge computing entity, and/or wherein the at least one local medical entity and the central computing entity are configured to communicate in real-time to provide the medical support function.

3. The medical support system of claim 1, wherein the central computing entity includes at least one artificial intelligence module configured to analyze the anonymous preprocessed data (APD) using an artificial intelligence algorithm.

4. The medical support system of claim 3, wherein the artificial intelligence algorithm is a machine learning algorithm, in particular a neural network.

5. The medical support system of claim 4, wherein the artificial intelligence algorithm is trained based on anonymous patient data.

6. The medical support system of claim 1, wherein the central computing entity is configured to provide a software component to the at least one local medical entity, and wherein the at least one local medical entity is configured to generate the anonymous preprocessed data using the software component.

7. The medical support system of claim 6, wherein the at least one local medical entity is configured to request the software component from the central computing entity according to a medical support function to be provided to the patient.

8. The medical support system of claim 1, wherein the at least one local medical entity is configured to encrypt the anonymous preprocessed data and send the encrypted anonymous preprocessed data to the central computing entity, and/or wherein the central computing entity is configured to encrypt the one or more parameters and send the encrypted one or more parameters to the at least one local medical entity.

9. The medical support system of claim 1, wherein the at least one communications network includes at least one of a local network and a mobile network, in particular a 5G mobile network, and/or wherein a latency of the at least one communications network is 10 ms or less, and/or wherein a bandwidth of the at least one communications network is 100 Mbit/s or higher.

10. The medical support system of claim 1, wherein the medical support function is selected from the group consisting of medical diagnostics, medical decision-making, medical treatment of the patient, and combinations thereof.

11. The medical support system of claim 1, wherein the medical support system is configured for cardiac disease diagnostics, cardiac disease treatment, neurological treatment, electrophysiology, medical virtual reality procedures, medical enhanced reality procedures, robot-assisted surgery, and combinations thereof.

12. The medical support system of claim 1, wherein the anonymous preprocessed data include one or more technical parameters derived from the physiological data of the patient and relating to an execution of the medical support function.

13. The medical support system of claim 1, wherein the one or more parameters determined by the central computing entity are procedure control parameters for controlling the medical support function, in particular wherein the procedure control parameters are determined based on the one or more technical parameters provided by the at least one local medical entity.

14. Medical support method for patient treatment, comprising:
acquiring, by at least one local medical entity, physiological data of a patient, preprocessing, by the at least one local medical entity, the physiological data to generate anonymous preprocessed data,
sending, by the at least one local medical entity, the anonymous preprocessed data to a central computing entity via at least one communications network,
analyzing, by the central computing entity, the anonymous preprocessed data,
determining, by the central computing entity, one or more parameters in relation to a medical support function based on the analysis of the anonymous preprocessed data, sending, by the central computing entity, the determined one or more parameters to the at least one local medical entity via the at least one communications network, and implementing, by the at least one local medical entity, the one or more parameters received from the central computing entity to provide the medical support function for the patient.

15. A machine readable medium comprising instructions executable by one or more processors to implement the medical support method according to claim 14.
